(19) United States
(12) Patent Application Publication
(10) Pub. No.: US 2002/0110523 A1
(43) Pub. Date: Aug. 15, 2002

(54) Methods for screening or monitoring the risk of cardiovascular disease relating to sex steroid compound or composition intake and methods for screening sex steroid compound

(76) Inventors: Cornelis Kluft, Sassenheim (NL); Josephus Jan Emeis, Boskoop (NL)

Correspondence Address: NIXON & VANDERHYE P.C., 8th Floor, 1100 North Glebe Road, Arlington, VA, 22201-4714, US

(21) Appl. No.: 09/985,561

(22) Filed: Nov. 5, 2001

Related U.S. Application Data
(62) Division of application No. 09/254,348, filed on Mar. 23, 1999.
Application No. 09/254,348 is a 371 of international application No. PCT/NL96/00350, filed on Sep. 6, 1996.

(51) Int. Cl. C12Q 1/00; A61K 49/00

(52) U.S. Cl. 424/9.2; 435/4

(57) ABSTRACT

A method for screening for negative side effects of a sex steroid compound or composition in a subject, by carrying out an assay on the subject or on a sample derived from the subject determining whether an increase of the compound or composition on the level of an acute phase reactant or a metabolic derivative thereof has occurred since applying the compound or composition to the subject, the acute phase reactant being selected from the group consisting of positive Acute Phase Reactants (ARPs) with the exclusion of ceruloplasmin and coagulation/thrombosis associated factors. An increase in the level of the acute phase reactant being indicative of negative side effects. A sex steroid compound or composition characterized by a lower increase in APR level than a third generation oral contraceptive, as determined in a manner according to the invention is also provided; the compound or composition not being a second generation oral contraceptive.

Preparations with sex steroids have been developed for birth control, supplementation e.g. when women enter the menopause, replacement when women have had hysterectomies. Alternatively they are used for sex-change processes and growth inhibition for tall boys and girls.

The preparations used in the field of anticonception have in recent history been adapted with regard to both dosage and type of compounds. The reason was the recognition that besides the targeted action of the sex steroids in anticonception, additional effects occurred in the first generation pills and increased occurrence of cardiovascular disease in these groups was documented in epidemiological surveys. The dose of the estrogenic component was identified as an important determinant and was reduced gradually with time. Up till recently, epidemiology supported these adaptations of the third generation in showing reductions in cardiovascular side affects. Newer formulations with lower dosages are being evaluated for future use. Up till recently, epidemiology supported these adaptations in showing reductions in cardiovascular side effects.

Epidemiological studies are preceeded by studies on intermediary end-points or beacons to document before introduction of a new formulation with the promise of reduced side effects. Evaluations of intermediary end-points for cardiovascular disease have focussed on metabolic changes in lipid, carbohydrate and haemostatic processes. In more recent years their usually separate evaluation was more integrated as advocated by the consensus development meeting on this topic in Esbjerg, Denmark held from May 18-20 in 1989. The said consensus also concluded that the metabolic changes followed the trend of being smaller with newer pills, and that the objective to be achieved in future is a minimal change or even more desirably absence of any change in metabolism. The consensus also stated “The metabolic alterations induced by progestagen-only pills were not considered extensively because current evidence suggested that changes are small even by sensitive indicators of lipid and carbohydrate metabolism and components of hemostasis. Furthermore there are insufficient epidemiologic data to determine whether the progestagen-only pills influence the risk of cardiovascular diseases.” They further state that the progestagen component is mainly responsible for the effect of OCs (oral contraceptives) on carbohydrate metabolism and that the effects on hemostasis are conceivably estrogen mediated because they have not been demonstrated in progestagen-only preparations.

Oral contraceptives most often contain both an estrogenic and a prostagenic compound. The mechanisms involved in metabolic changes to date were considered to reflect estrogenic metabolic effects of the estrogen component and, depending upon the compound, intrinsic estrogenic, androgenic or anti-estrogenic properties of the progestagenic component. Firstly with this in mind the dosage of estrogen was lowered. In addition with this in mind a set of modern, new prostagenic compounds including desogestrel, gestodene and norgestimate were in recent years introduced and they represent the so-called third generation Ocs. The newer progestins were designed to minimize the adverse effects (e.g., acne, hirsuitism, nausea, carbohydrate and lipid metabolism changes) observed with older Ocs, while maintaining efficacy and good menstrual cycle control. Desogestrel, norgestimate, and gestodene have minimal amounts of androgenicity and antiestrogenic potential as described in Ann-Pharmacother. July-August 1995; 29(7-8): 736-42. This article reviews and compares the newer progestins desogestrel, norgestimate, and gestodene with regard to chemistry, pharmacokinetics, efficacy, and tolerability. This article was based on primary literature on desogestrel, norgestimate, and gestodene as identified from a comprehensive MEDLINE English-literature Harch from 1984 through 1994, with additional studies selected by review of the references. Indexing terms included progestins, desogestrel, gestodene, norgestimate, levonorgestrel, and norgestrel. Only human clinical and pharmacokinetic trials performed in Europe, Canada, and the US were included. All available data from human studies were reviewed; both comparative and noncomparative studies were included because of the paucity of direct comparative information available. Desogestrel, norgestimate, and gestodene show minimal amounts of androgenicity and antiestrogenic potential. All of these agents are pharmacokinetically similar to older agents: they are highly bioavailable when administered orally, hepatically metabolized, and obtain steady-state concentrations after 8-10 days of continuous administration. The newer agents have similar Pearl Indexes and slightly better cycle control. Furthermore, the new progestins appear to cause fewer adverse effects, such as acne and hirsuitism, and similar rates of weight gain, blood pressure changes, and lipid and carbohydrate metabolism changes. The conclusion of this article was: “Desogestrel, norgestimate, and gestodene appear to offer clinical advantages because of their decreased androgenicity. Women whose cycles are currently well controlled with other OCs should not be switched to a newer progestin. However, any of the combination OC products that contain these progestins may be prescribed for women intolerant of older agents” or to first-time users of OCs. The newer progestins appear to be efficacious and offer similar cycle control, improved safety and tolerability profiles, and comparable price with the older agents. A comparative evaluation of the androgenicity of four low-dose, fixed-combination oral contraceptives described in Int. J. Fertil. Menopausal Stud. 1995; 40 Suppl 2; 92-7 by C. M. Coenen, C. M. Thomas, G. F. Borm and R Rolland describes how changes in endogenous androgen metabolism were compared in healthy women taking one of four low-dose modern oral contraceptives (OCs). One hundred women were randomized to (1) 35 micrograms ethinyl estradiol (EE)+250 micrograms norgestimate (Cilest); (2) 20 micrograms EE+150 micrograms desogestrel (Mercilon); (3) 30 micrograms EE+150 micrograms desogestrel (Marvelon); or (4) 30 micrograms EE−75 micrograms gestodene (Femodene). During the luteal phase of the pretreatment cycle, body weight and blood pressure were recorded, and plasma levels of the following variables were recorded: sex-hormone-binding globulin (SHBG), cortisol-binding globulin (CBG), testosterone, free testosterone, dihydrotestosterone, androstenedione, dihydroepiandrosterone sulfate (DHEAS), and hydroxyprogesterone. The free androgen index was also calculated. These variables were remeasured during the third week of OC intake and during the fourth and sixth cycles. There were no statistically significant differences in androgenic variables among the four OCs. The DHEAS concentration decreased less with the 20 micrograms EE+desogestrel formulation compared with either 30 micrograms EE+desogesterel or norgestimate-containing formulations (20% vs. 45%). Concentrations of SHBG and CBG increased significantly in all four groups (average 263+/−119% and 94 +/−26%, respectively); CBG increased less in women taking 20 micrograms EE+desogestrel (about 75%) than in the other formulations (about 100%). The four modern, low-dose OCs tested had similar impacts on endogenous androgen metabolism, yielding significant decreases in testosterone, dihydrotestosterone, androstenedione, and DHEAS. All of these formulations were considered potentially beneficial in women with androgen-related syndromes such as acne and hirsuitism. Implementation of large studies to establish which of the third-generation OCs is the least androgenic is described. In vitro studies suggested that norgestimate had the least androgenic profile according to this article.

Markers of the steroid effect used to date in assessing the net effect of combined preparations better, and more specifically to evaluate the estrogen dominance and androgenicity are the sex hormone binding globulin (SHBG), cortisol-binding globulin (CBG), testosterone, free testosterone, dihydrotestosterone, androstenedione, dihydroepiandrosterone sulfate (DHEAS), the free androgen index, and ceruloplasmin.

The general way of thinking has been to consider changes in the metabolic systems as a consequence of direct or indirect effects on synthesis or clearance of the component under study and some sex steroid responsive elements have already been identified for a number of these factors. This way of thinking also dominates for factors that can be regulated by other mechanisms. An example concerns fibrinogen which can be upregulated by inflammatory processes and is also involved in the clotting process. E. Ernst amoung others has correlated fibrinogen increases following use of oral contraceptives to the estrogenic compound and thus derived an increased risk of cardiovascular disease (Atherosclerosis 93 (1992) 1-5. A. Ruokonen and K. Kaar (Europ. J. Obstet. Gynec. reprod. Biol. 20 (1985) 13-18) have tested the effects of desogestrel, levonorgestrel and lynestrenol i.e. representatives of generations 1-3 of oral anticonceptive progestagens on SHBG, cortisol binding protein (CBP), ceruloplasmin and HDL-cholesterol. They concluded lynestrenol was weakly estrogenic in man but could find no estrogenic effect for the other two progestagenic compounds. All three compounds were concluded to have androgenic activity on the basis of a change in SHBG and HDL cholesterol levels. Of the three compounds desogestrel which is the third generation product was found to be the least androgenic.

The role of estrogenic effects on a haemostatic factor has also previously been indicated through correlation of changes in blood concentrations of said haemostatic factor under study with changes in ceruloplasmin.

Recent epidemiological reports have described increased thrombosis in users of the third generation compositions. These new epidemiological data came as a complete surprise since till that time all intermediary risk indicators and beacons used so far to judge the third versus second generation indicated a safer profile of the third generation. The recent international consensus meeting on the metabolic effects of oral contraceptives in Amsterdam (November 1995) concluded that reliable beacons had concomitantly been lost and nobody knew what was next. The statement has been published in Gynaecol. Endocrinol. 10 (1996) 1-5. The subject invention aims to solve this problem by providing new and reliable markers.

The subject invention is thus directed at a method for screening for negative side effects of a sex steroid compound or composition by assessing the level of a marker selected from a group of proteins or complexes of proteins hitherto never associated with a sex steroid compound or known negative side effects thereof in a subject after said subject has been dosed with said compound or composition. A sex steroid composition is a composition useful for the applications described above in particular as contraceptive for reproduction and in hormone replacement or supplementing therapy. Specifically a sex steroid composition comprises an oestrogenic and/or progestogenic compound or combination thereof. By using this screening method it has now become possible to select a compound or a combination of compounds that is safe to use. In particular by use of this screening method a compound or composition having a lower risk than the third generation compounds for cardiovascular disease can be found and even preparations with a lower risk than the second generation pills. We can even progress to compounds or compositions exhibiting no risk. Compounds or compositions that exhibit an increased or high risk can be determined and subsequently rejected for further research at an early stage in development of alternative sex steroid preparations such as anticonceptives and hormone replacement or supplementing compositions. It is also possible to carry out the method according to the invention in order to assess the impact of a particular sex steroid preparation such as a contraceptive on the subject taking it in order to monitor potential problems arising from taking the preparation at any time after taking the preparation as a control assay. This can be particularly useful in view of the fact that risks associated with oral contraceptives to date appear to be highest shortly after commencing intake. This is apparent from the articles

N R Poulter, T M M Farley, C L Chang, M G Marmol, O Meirik. Safety of combined oral contraceptive pills. The Lancet 1996; 347: 547.

H M B Reynen, W I Atsma

Risk is highest during first month of use. Letter to the Editor. B. Med. J 1995; 311: 1639. Preferably therefore a method according, to the invention can be applied within the first one to six months of intake of the preparation in order to assess whether the subject is sensitive to the preparation.

In a recent study on oral contraceptives with 30 ug Ethinylestradiol and either gestodene or desogestrel as progestagen we included as a specific non-lipid, non-carbohydrate, non-haemostatic variable a control protein. This protein was a protein for the acute phase reaction: C-reactive protein (CRP). We were subsequently highly surprised to observe a strong increase in this component during use of both oral contraceptives. There was no apparent reason to have an acute phase response of such a magnitude in nearly all the young volunteer women. We confirmed an even larger increase in CRP in a similar study with similar oral contraceptives with 20 ug EE.

The group of acute phase reactants (APR) has been defined by Thompson et al (D. Thompson, A Milford-Ward and J. T. Whicher in Ann Clin. Biochem. 1992; 29:123-131) and consists of plasma proteins which can increase in concentration by 25% or more in the first 7 days following tissue damage.

The article by I. Kushner and A. Mackiewics in Disease markers 1987; 5:1-11 and articles by Steel et al and Thompson et al (cited elsewhere in the description) provide details of acute phase reactants as defined in the art. The acute phase of the inflammatory response refers to a wide range of physiological changes initiated immediately after infection or physical trauma. The ensuing cascade of mediators induces activation proliferation and altered behavior and changes in the biosynthetic capacities of a variety of target cells and tissues. An important aspect of the acute phase response is the radically altered biosynthetic profile of the liver. Hepatic acute phase reactants comprise the following proteins:

complement proteins: C2, C3, C4, C5, C9, Factor B, C1 inhibitor, C4 binding protein,

proteinase inhibitors: α1-antitrypsin, α1-antichymotrypsin, α2-antiplasmin, Heparin cofactor II,

metal binding proteins: haptoglobulin, hemopexin, ceruloplasmin, manganese superoxide dismutase,

major acute phase reactants: serum amyloid A, C reactive protein, serum amyloid P,

coagulation related proteins: fibrinogen, von Willebrand Factor, plasminogen activator inhibitor I,

other proteins: α1-acid glycoprotein, heme oxygenase, mannose binding protein, leukocyte protein I, lipoprotein (a), lipopolysaccharide binding protein

negative reactants: albumin, pre-albumin, transferrin. ApoA1, ApoAII, α2-HS glycoprotein, inter α-trypsin inhibitor, histidine-rich glycoprotein.

A number of other reactants also exist such as the major reactant Secretory Phospholipase A2 (sPLA2), ferritin, retinol binding protein, and coagulation related proteins i.e. tissue plasminogen activator and plasminogen. A number of proteins have also been proposed to belong to the group of APRs but remain to be confirmed. This group comprises angitensinogen, kininogen, kininogenase, fibronectin, prothrombin, Factor VIII, heparin cofactor II. Some of the hepatic reactants have also been illustrated to be produced extra hepatically e.g. SAA.

The acute phase reactants vary in degree in which they are or can be activated. In general an acute phase reactant is defined as a reactant that can vary in concentration by 25% or more in the first 7 days of inflammation or following tissue damage. The major acute phase reactants can be produced up to 1000 fold their basic level. Ceruloplasmin and C3 exhibit about 50/% increase.

The response is known to be initiated and coordinated by a large number of inflammatory mediators, which include cytokines, anaphylatoxins and glucocorticoids. Several hormones specifically regulate the transcription of human APRs. These include interleukin 1, interleukin 6, tumor necrosis factor, transforming growth factor β, interferon gamma, glucocorticoids and effector molecules comprising interleukin 2, oncostatin M, ciliary neurotrophic factor and retinoic acid. In particular interleukin 1 and tumor necrosis factor induce APR synthesis in the liver and stabilisation of mRNA for positive APRs occurs by cytokines and glucocorticoids. Mackiewicz A., Ganapathi M. K., Schultz D. and Kushner I. describe in J.Exp.Med (1987) 166:253-258 how cytokines may regulate alterations of glycosylation of some cytokines with consequences for their activity and stability. Interleukin 1β and interleukin 6 can both induce SAA mRNA and protein synthesis (Steel et al cited elsewhere).

In our experiment using CRP as an indicator of inflammatory processes, we further observed that the OC-induced increases in fibrinogen and ceruloplasmin were correlated to a compatible degree with their status as acute phase reactants of the liver so called APP, acute phase proteins. In addition we determined the endothelial cell factor von Willebrand factor was associated to a degree compatible with its increase in association with acute phase reactions. It was concluded that both liver and endothelial cell factors that can react to inflammation with a so-called acute phase response were involved.

In an animal experiment with rats we further confirmed the vascular effect on APR's von Willebrand factor and fibrinogen known to be haemostatic/thrombotic factors. We could not however test for CRP in these animal experiments because this protein is absent in rats. Surprisingly, in these rat experiments we furthermore ascertained that the effects were confined to two OCs of the third generation and did not occur with a second generation OC.

These data suggest that the pro-inflammatory or acute phase effects on the vascular wall are stronger in the third generation OCs an effect which has, however until now never been identified in review papers or by authors of such publications. Upon our own literature re-evaluation of effects on von Willebrand factor and the related factor VIIIc assay, no direct comparison was available. Reading different reports with the new insight described above the trend of increase in von Willebrand factor with only the third generation OCs in humans could in retrospect be found. This supported our hypothesis that depending upon the composition of OCs a quantitatively different pro-inflammatory aspect had to be assumed. Theoretically such a pro-inflammatory effect can contribute to increase of trauma and therefore to an increased risk of thrombosis in cases sensitive to these changes in risk. The increase in CRP is most prominent of the proteins tested. Its postulated role of pro-inflammatory component itself, and in increasing tissue trauma effects makes an increase in this component undesirable for persons defective or weak in control mechanisms of trauma reactions such as individuals with hereditary defects in the anticoagulation system (deficiencies in antithrombin III, protein S and Factor V Leiden). We have now ascertained that an increase in CRP not only marks acute phase effects but also adds directly to the risk of venous thrombosis. The increase in the vascular von Willebrand factor and in factor VIII itself are also considered undesirable in view of the previously already described association between elevated levels of these components and the risk of venous thrombosis (Br-Heart-J. December 1995 ; 74(6): 580-3). This article describes that upon release, vWF seems to mediate platelet aggregation and adhesion to the vascular endothelium. It further states because the release of vWF is increased when endothelial cells are damaged, vWF has been proposed as an indicator of endothelial disturbance or dysfunction. The availability of such an index of endothelial dysfunction is postulated as having clinical value, because measurement of such a marker can be a non-invasive way of assisting in diagnosis or as an indicator of disease progression. The known association between vWF, thrombogenesis, and atherosclerotic vascular disease also suggests that high concentrations of vWF may be an indirect indicator of atherosclerosis and/or thrombosis. In addition, high vWF concentrations have prognostic implications in patients with ischaemic heart disease and peripheral vascular disease. We conclude that the increase in von Willebrand factor may not only be deleterious in itself in the manner disclosed above, but also may reflect other changes in endothelial functioning as now discovered by us. Von Willebrand factor was already a broadly accepted indicator of endothelial dysfunction but it can now be used as beacon or intermediary risk indicator in a broader sense for assessing the risk associated with Ocs.

It should be remarked that the increase of trauma-reactions with the third versus the second generation of OC, correlates with the recent epidemiological reports of increased thrombosis in users of the third generation. These new epidemiological data came as a complete surprise since till that time all intermediary risk indicators and beacons used so far to judge the third versus second generation indicated a more safe profile of the third generation. Our identification of the mechanism of increased/reinforced trauma is unexpected and new. It implies that OC formulations as well as other steroid drugs should be selected for minimal or no impact on inflammatory processes such as can be monitored by relevant liver factors (e.g. CRP) as such or in combination with vascular factors (e.g. von Willebrand factor). In the case of OCs the role of the third generation progestagens seems to dominate.

Further studies have revealed that some acute phase reactants are more suitable than others to be used as markers in a screening assay according to the invention due to the degree in which they are affected. The group of APRs (acute phase reactants) exhibiting more than 100 fold incremental change during an acute phase reaction forms an extremely suited group of proteins for carrying out a test according to the invention due to the high degree of sensitivity that can he achieved. This is in contrast to other proteins belonging to the group of APRs in general like ceruloplasmin, von Willebrand factor. Factor VIII or fibrinogen which exhibit much smaller increments.

The invention comprises a method for screening for negative side effects of a sex steroid compound or composition in a subject, by carrying out an assay on the subject or on a sample derived from the subject determining whether an increase of the compound or composition or the level of an acute phase reactant or a metabolic derivative thereof has occurred since applying the compound or composition to the subject, said acute phase reactant being selected from the group consisting of positive APRs with the exclusion of ceruloplasmin and the coagulation/thrombosis associated factors described above. A suitable group of embodiments of the above type comprises a method using an APR selected from metal binding proteins with the exclusion of ceruloplasmin. Another suitable group is that of proteinase inhibitors.

A preferred embodiment of the claimed invention is defined as follows: A method for screening for negative side effects of a sex steroid compound or composition in a subject, by carrying out an assay on the subject or on a sample derived from the subject determining whether an increase of the compound or composition on the level of an acute phase reactant or a metabolic derivative thereof has occurred since applying the compound or composition to the subject, said acute phase reactant being selected from the group consisting of APRs defined as being capable of exhibiting more than 100 fold increment within 7 days after tissue damage or inflammation, preferably more than 200 fold, whereby an increase in the level of the acute phase reactant is indicative of negative side effects.

The preferred markers to be used in the invention belong to the group of APRs known to be capable of exhibiting up to 1000 fold increase during an acute phase reaction Markers belonging to. Additionally preferred markers according to the invention belong to the group of APRs exhibiting a low or slightly variable basic level thus enabling good quantification of a disturbance in this level. This group comprises the proteins serum amyloid A (SAA), serum amyloid P (SAP), C-reactive Protein (CRP) and Secretory Phospholipase A2 (sPLA2) as disclosed by D. M. Steel and A. S. Whitehead in Immunology Today 1994; 15: 81-88 by Kushner et al as mentioned elsewhere in this description. Metabolic derivatives of such proteins such as CRP complex with complement system C3 of C4 can also be determined in an alternative embodiment. This can be assessed as disclosed by Wolbink G J, Brouwer M C, Buysmann S, ten Berg I J M, Hack C E, in Jo Immunol 1996; 157: 473-479. The derived products C4b, C4d, C3b and C3d can be assessed. The cited article is incorporated herein by reference. Additionally the increase in ARP level can be a decrease when the group of APRs is selected to consist of the negative APRs as disclosed elsewhere in the description.

The sample to be analysed in any of the embodiments of a method according to the invention can be a blood sample or any other sample suitable for assessing levels of plasma proteins as is common knowledge for a person skilled in the art. The method according to the invention can be carried out in an in vitro system or in an in vivo system. Both animal and human test systems can be used. CRP cannot be tested in a rat system however as it is absent in rats. Alternative APP proteins of the type described can however be used in a rat model. SAP is the rat homologue of CRP. CRP is one of the preferred markers for use in humans.

The detection methods to be used will depend on the marker to be analysed. Numerous assays are already known for determining the amounts of such proteins. By way of example the CRP can be determined using the CRP assay disclosed in the WHO Expert Committee on Biological Standardisation (37th Report, World Health Organ Tech. Rep. Ser. 1987; 760: 21-22 and New Engl. J. Med. 1994; 331: 417-424). The SAA assay can be carried out as disclosed by J. Wilkins, J. R. Gallimore, G. A. Tennent et al in Clin Chem 1994; 40: 1284-1290. SAP is described by R. L. Meek, S. Urieli-Shovall and E. P. Benditt in PNAS (1994): 91: 3186-3190. The C3 and C4 assays are described above. The protocols for these assays in the cited documents are hereby incorporated by reference. The person skilled in the art will be able to find sufficient references to enable performance of the relevant assay once the marker protein has been selected.

In a preferred method according to the invention the screening will additionally involve determination of the level of a haemostatic factor, said haemostatic factor also belonging to the group of APRs. Such factors are fibrinogen (Thompson et al cited above), von Willebrand Factor (B. E. Pottinger, R. C. Read, E. M. Paleolog, P. G. Higgins and J. D. Pearson in Thromb. Res. 1989; 53: 387-394). Tissue Plasminogen Activator (t-PA) (J. H. Jansson, B. Norberg, T. K. Nilsson in Clin. Chem 1989; 35: 1544-1545). Plasminogen Activator Inhibitor (PAI-I) (J. H. Jansson, B. Norberg, T. K. Nilsson in Clin. Chem 1989; 35: 1544-1545), plasminogen (I. Juhan-Vague, M. C. Alessi, P. Joly, X. Thirion, P. Vague, P. J. Declerck, A. Serradimigni and D. Collen in Arterisclerosis 1989; 9: 362-367) and α₂-antiplasmin (Steel et al as cited above). In particular a method for determining an increase in von Willebrand factor in combination with any of the preferred embodiments described above for the method according to the invention with the first APR falls within the preferred scope of the invention. Determination of an increase of an APR from the group defined as capable of exhibiting more than 100 fold increase within the first 7 days after tissue damage or upon inflammation provides a suitable embodiment of the aforementioned screening method with 2 APRs. Screening assays for haemostatic factors are described in large detail in the prior art. Specifically reference is made to the ECAT Assay Procedures. A manual of Laboratory techniques, European Concerted Action on Thrombosis and Disabilities of the Commission of the European Communities, edited by J. Jespersen. R. M. Bertina and F. Haverkate. The information regarding hamostatic factor assays of the factors described in the previous sentence is hereby incorporated by reference. Numerous alternatives exist and will be readily apparent to a person skilled in the art.

An alternative embodiment of the invention comprises an in vitro method for screening for negative side effects of a sex steroid compound or composition in a subject, by carrying out an assay on a sample that has been exposed to the compound or composition determining whether an increase of the compound or composition on the level of an acute phase reactant modulator or a metabolic derivative thereof has occurred since applying the compound or composition. The modulator being suitably selected from the group consisting of biological response modifiers. Such biological modifiers comprise the modifiers mentioned earlier contributing to induction of APRs such as cytokines, interleukins etc. Specifically the enhancers of CRP are preferred.

We hypothesize that estrogens appear to have a stimulatory effect on a package of acute phase proteins. This stimulatory effect is counteracted by levonorgestrel thereby providing a good combination pill upon assessment of the results derived from our experiments in combination with previously published literature of Roukonen and Kaar (cited elsewhere in the description). With desogestrel the effect of estrogen is not countered and thus this combination is less successful and according to our hypothesis causes more thrombosis than the second generation pills.

In order to provide an improved sex steroid preparation exhibiting less or even no negative side effects in the future the following steps can be taken:

1) Screening for an estrogenic variant that does not stimulate the acute phase reactants.

2) Screening for a progestogen variant or other compound that inhibits the acute phase activation of estrogen and/or inhibits the basic acute phase reaction and preferably at least to a degree sufficient to negate the acute phase reaction. Levonorgestrel is an example of such an inhibitor which can probably be improved further.

In particular the negative side effects to be combatted are those of increased risk of cardiovascular disease such as thrombosis. In more general terms the negative side effects are those correlated to increase in one or more acute phase reactants. The screening can occur using any of the methods according to the invention as disclosed above. The compounds thus screened can be novel compounds or can be found amongst known compounds. Combination of the compounds can lead to a sex steroid preparation as safe as the third generation pills or better. It can also lead to alternative formulations at least as good as the known second generation pills. The determining factor is that the new formulation comprising either a novel compound or a combination of novel compounds or a novel combination of known compounds exhibits less increase in an APR than a third generation pill preferably less than a second generation pill when these are compared. In vitro tests can provide the possibility to carry out such assessments under equivalent conditions. Preferably a sex steroid preparation according to the invention will not result in an increase of an APR as can be determined using a method according to the invention.

On the basis of the results obtained in our experiments the negative side effects of sex steroid preparations as indicated by an increase in APR value in any of the methods according to the invention can be reduced by a non oral administration route. This is due to the fact that most of the APRs are hepatically produced and oral application of hormonal medication is known to provide the largest exposition of the applied compound to the liver. Sex steroid preparations which upon oral application fail to pass the test according to the invention on the basis of their effect on APR levels could be applied via topical application to the vagina or via the skin e.g. by using a plaster or a cream and result in acceptable APR levels. Another embodiment comprises subcutaneous use of implants as secretory devices, secretory intrauterinary devices or injection. Such alternative forms of providing medication are as such known for other fields of medicine and for some sex steroid preparations. Examples of known intrauterine devices releasing a sex steroid preparation are the Progestasert which releases progesterone at a rate of 65 micrograms per day and the LNG-20 which releases 20 micrograms of levonorgestrel per day. The Norplant system employs silastic tubing permeable to steroid molecules to provide stable circulating levels of levonorgestrel. Depot-medroxyprogesterone acetate (Depo Provera) is given in the form of an injection as is norethindrone enanthate. Transdermal application of oestrogen is also known.

Naturally the embodiments causing least discomfort and ease of application will be preferred. A number of the above alternatives are not acceptable for a number of reasons which are well documented in the literature. We refer in particular to Part III Contraception, Chapters 22 and 23 a handbook. It is to be expected that oral application will remain the favorite mode of application. Development of novel oral sex steroid preparations using the method according to the invention is one of the main objectives of the subject invention. However development of alternative embodiments as disclosed above is also an objective as they also can overcome the higher risk associated with sex steroid preparations taken orally.

The following examples illustrate and support the invention. The invention is not restricted to the examples.

### EXAMPLE I

Thirty-nine apparently healthy female subjects aged 20 to 40 years who had regular menstrual cycles (21 to 35 days) and not using OC or being pregnant the previous three month were enrolled in this study. None of the routine hematologic and serum-chemical screening and urine analysis showed abnormal results. Informed consent was obtained from all subjects and the study was approved by the Committee on Medical Ethics of the University of Leiden.

Fasting blood samples were taken between 8.00 and 10.00 AM during the 17th and 25th day of the pretreatment cycle and between the 18th and 21 st day of the 3rd, 6th, and 12th cycle on OC.

The volunteers were randomized (open label) to the use of two different OC preparations with an equal dose of ethinylestradiol (30 ug), but different type and dose of progesteron, being either 75 ug gestodene (GTD-EE) or 150 ug desogestrel (DSG-EE)

C-reactive protein was measured with an enzyme-immuno-assay, using horseradish peroxidase labelled polyclonal antibodies against CRP (Dako, Copenhagen, Denmark) as catching and tagging antibody in a manner known per se. CRP standard serum (Behringwerke, Marburg, Germany) was used to standardize the results. The detailed procedure was as follows.

CRP Assay

Plasma CRP levels were measured by an enzyme immunoassay (ELISA) . The microtitration plates were coated with 100 μl Rabbit-anti-human CRP (DAKO, Denmark) in coating buffer (1:8000) and incubated overnight at 4° C. The wells were washed three times with 0.1% PBS-Tween-20 and ImM Na₂EDTA. The plasma samples diluted 1:1000 (Hamilton, MicroLab 1000) in PBS-Tween containing 3% polyethylene glycol 6000 were added in duplicate to the coated wells. The plates were incubated for two hours at room temperature, the wells were rewashed three times and 100 μl peroxidase-conjugated rabbit-anti-human CRP was added for one hour at room temperature. After washing three times 100 μl TMB solution was added. The colour reaction was stopped with 4 M sulphuric acid and the extinction was measured by reading at 450 nm in a Titertek multiscan photometer. The standard line (0.08-21.5 ng/ml) was made of CRP standard serum (Behring, 7.6 mg/dl)

For further details reference is made to Chapter 32 of J. A. Gevers Leuven et al in the book Fibrinolysis in Disease, Molecular and Hemovascular Aspects of Fibrinolysis edited by Pia Glas-Greenwalt, for what was analysed further and the used methods; From the sixty-nine from the Gevers Leuven study we only studied 39 for CRP.

Observations:

| | CRP-values | | | | |
|---|---|---|---|---|---|
| | Pre- | | | | cy- |
| | treatment | 3rd | 6th | 12th | cle |
| GTD- | | | | | |
| EE | | | | | |
| Number | 19 | 19 | 18 | 18 | |
| Median | 0.260 | 1.170# | 0.750 | 2.075 | |
| IR | 0.13-0.73 | 0.83-2.04 | 0.488-1.538 | 0.98-2.483 | |
| DSG- | | | | | |
| EE | | | | | |
| Number | 20 | 20 | 18 | 19 | |
| Median | 0.190 | 0.785# | 0.740* | 1.310# | |
| IR | 0.113-0.560 | 0.353-1.422 | 0.435-2.093 | 0.550-2.030 | |

IP = interquartile range: Wilcoxon Matched-Pairs signed-ranks test compared to preatreatment,
*= p < 0.05;
#= p < 0.005.

It was concluded that the use of both Ocs induced an increase in CRP. The increase lasted at least for 6 cycles. The increment in CRP from pretreatment to the third cycle showed a trend of being larger for GTD-EE (p=0.063).

### EXAMPLE II

In the pretreatment samples of example I (n=38) a very strong correlation (Spearman) between CRP and ceruloplasmin (r=0.664, p<0.0005) was further identified in agreement with the known acute phase character of ceruloplasmin. In contrast to Ruokonen and Kaar (cited above) who regard ceruloplasmin purely as an indicator of oestrogenic activity of Ocs we chose to regard ceruloplasmin primarily as a powerful acute phase indicator. In agreement herewith a correlation of ceruloplasmin with another plasma component with an acute phase character was identified: fibrinogen (r=0.3264; p=0.012, n=58).

The change in CRP induced by OC-use (example I) was also correlated with a change in ceruloplasmin. For both Ocs or example I taken together (n=39) the increment in CRP correlated with a Spearman coefficient of 0.4839 (p=0.002) with an increment in ceruloplasmin after three cycles.

For all three sampling moments during OC-use (example I; combining both OCs) the changes in ceruloplasmin compared to the pretreatment situation also correlated with the changes in fibrinogen (r=0.3143; 0, 4386, 0, 4576 (n=58). In addition the changes in von Willebrand factor and the closely related variable, Factor VIIIc. correlated with the changes in fibrinogen at all sampling moments (r=0.3185 to 0.4751)(n=58).

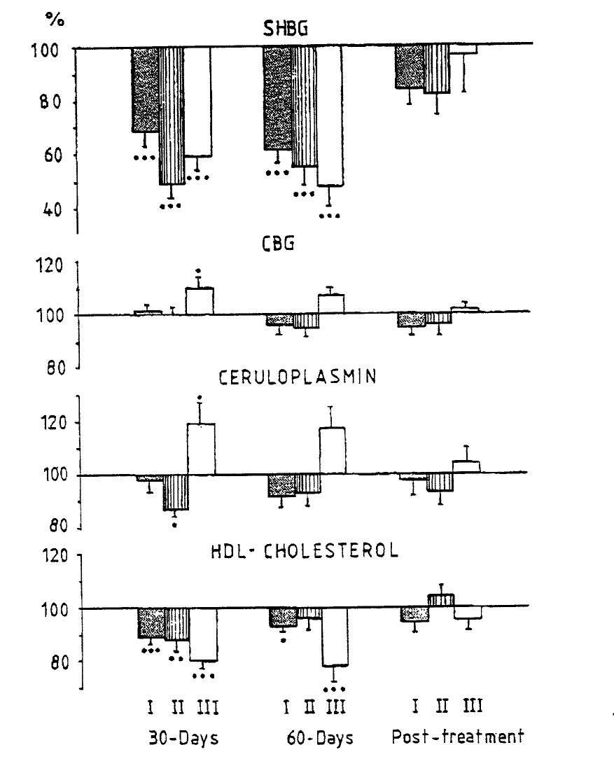

The conclusion was that OCs apart from inducing changes in CRP also induce related changes in other acute phase reactants either originating from the liver (APP e.g. ceruloplasmin) or the vessel wall (APP e.g. von Willebrand factor). When using this insight to analyse in retrospect FIG. 1 of Ruokonen and Kaar (cited above) it is apparent levonorgestrel causes the ceruloplasmin concentration to decrease whereas desogestrel does not induce this effect or does so to an insufficient degree. FIG. 1 illustrates:

thrombosis predominantly occurs in the first few cycles of third generation pills. Levonorgestrel decreases ceruloplasmin specifically in the first cycle

lynestrenol appears to be undesirable.

We feel FIG. 1 could be interpreted as a suppression of the low-grade acute phase as basically present in women by levonorgestrel. This provides a strong argument with regard to the recently discovered problem of the third generation pills in comparison to the second generation pills and our hypothesis concerning stimulatory effect on a package of acute phase reactants.

### EXAMPLE III

The effect of estrogen replacement therapy on CRP blood concentrations in 40 postmenopausal women with type 2 diabetes was studied in a randomized placebo-controlled trial. Twenty patients were treated orally with 2 mg/d of micronized 17 beta estradiol and 20 patients were treated with placebo for six weeks. Age (range: 49-68 years) and body mass index (range: 18,9-41.1 kg/m2) did not differ between the groups. Fasting blood samples were taken at baseline and after 6 weeks. For analysis of CRP, samples were available from 19 subjects from the placebo group and from 16 from the treatment group. Informed consent was obtained from all subjects and the study was approved by the Committee on Medical Ethics of the University of Leiden.

C-reactive protein was measured with an enzyme-immuno-assay, using horseradish paroxidase labelled polyclonal antibodies against CRP (Dako, Copenhagen, Denmark) as catching and tagging antibody. CRP standard serum (Behringwerke, Marburg, Germany) was used to standardize the results (see detailed procedure according to example I). Observations:

CRP-values

| | Pretreatment | 6 weeks |
|---|---|---|
| Placebo | | |
| Number | 19 | 19 |
| Median | 1.602.11 | |
| IR | 0.98-5.09 | 0.80-4.99 |
| Treatment | | |
| Number | 16 | 16 |
| Median | 4.13 | 6.70# |
| IR | 1.09-10.568 | 2.313-16.025 |

IP = interquartile range; Wilcoxon Matched-Pairs signed-ranks test compared to pretreatment. # p = 0.0027. The pretreatment values in both groups are not statistical significant different.

We concluded that the use of 17-beta estradiol induced an increase in CRP. Note: It is also clear that these diabetic women already have relatively high levels of CRP, when compared to young women (see example I), healthy middle aged man (median 0.427; IR 0.144-0.776; n=46) and healthy males and females 65 years and over (median 1.38: IR 0.643-2.528; n=140) (not statistically tested). This however does not render the test impracticable.

1. A method for screening for negative side effects of a sex steroid compound or composition in a subject, by carrying out an assay on the subject or on a sample derived from the subject determining whether an increase of the compound or composition on the level of an acute phase reactant or a metabolic derivative thereof has occurred since applying the compound or composition to the subject, said acute phase reactant being selected from the group consisting of positive Acute Phase Reactants (APRS) with the exclusion of ceruloplasmin and coagulation/thrombosis associated factors, whereby an increase in the level of the acute phase reactant is indicative of negative side effects.

2. A method according to claim 1 wherein the APR is selected from the group of APRs being metal binding proteins.

3. A method according to claim 1 wherein the APR is selected from the group of APRs being proteinase inhibitors.

4. A method according to any of the preceding claims, wherein the acute phase reactant is selected from the group defined as capable of exhibiting more than 100 fold increment within 7 days after tissue damage or upon inflammation, whereby an increase in the level of the acute phase reactant is indicative of negative side effects.

5. A method according to any of the preceding claims, wherein the acute phase protein is selected from the group consisting of APR defined as capable of exhibiting up to 1000 fold increment within 7 days after tissue damage or upon inflammation.

6. A method according to any of claims 1, 4 or 5, wherein the acute phase reactant is selected from the group consisting of Serum Amyloid A (SAA), Serum Anyloid P (SAP), C-Reactive Protein (CRP) and Secretory Phospholipase a2 (sPLA2).

7. A method according to any of the preceding claims wherein the acute phase reactant is a plasma protein.

8. A method according to any of the preceding claims comprising assessing the increase in level of a metabolic derivative of the APR.

9. A method according to claim 8, wherein said metabolic derivative is the C3 or C4 complex of C-Reactive Protein (CRP).

10. A method according to any of the preceding claims, said method being carried out in vitro.

11. A method according to any of claims 1-9, said method being carried out in vivo.

12. A method according to any of the preceding claims being carried out on a animal non-human subject or sample derived from an animal non-human subject.

13. A method according to any of the preceding claims, said method being carried out with Serum amyloid P as APR.

14. A method according to any of claims 1-11 or 13, said method being carried out on a human subject or sample derived from a human.

15. A method according to any of claims 1-11, 13 or 14, said method being carried out with CRP as APR.

16. A method according to any of the preceding claims, said method further comprising determining whether an increase of the compound or composition on the level of a second acute phase reactant or a metabolic derivative thereof has occurred since applying the compound or composition to the subject, said second acute phase reactant being selected from the group consisting of APRs associated with haemostasis i.e. coagulation or thrombosis, whereby an increase in the level of the second acute phase reactant in addition to that of the first is indicative of negative side effects.

17. A method according to claim 16, wherein the second APP plasma protein is selected from the group consisting of factors associated with haemostasis i.e. coagulation or thrombosis e.g. fibrinogen, von Willebrand Factor (vWF), Tissue Plasminogen Activator (t-PA), Plasminogen Activator Inhibitor I (PAI-1), plasminogen and α₂-antiplasmin.

18. A method according to claim 16 or 17 wherein said second APP plasma protein is von Willebrand Factor.

19. An in vitro method for screening for negative side effects of a sex steroid compound or composition, by carrying out an assay on a sample exposed to the compound or composition determining whether an increase of the compound or composition on the level of an acute phase reactant modulator or a metabolic derivative thereof has occurred since applying the compound or composition.

20. A method according to claim 19 wherein the modulator is selected from the group consisting of biological response modifiers.

21. A method according to claim 19 or 20 wherein the modulator is selected from inflammatory mediators, which include cytokines, anaphylatoxins, glucocorticoids, interleukins e.g. 1 and 6, tumor necrosis factor, transforming growth factor β, interferon gamma, and effector molecules comprising interleukin 2, oncostatin M, ciliary neurotrophic factor and retinoic acid.

22. A method according to claim 21, wherein the modulator is selected from the group consisting of interleukin 1 and tumor necrosis factor, cytokines and glucocorticoids.

23. A method according to any of claims 19-22, wherein the modulator specifically enhances levels of CRP.

24. A method according to any of the preceding claims except claim 11, wherein said subject is a human being.

25. A method according to any of the preceding claims, wherein said sample is a blood sample.

26. A sex steroid compound or composition characterised by a lower increase in APR level as determined in a manner according to any of the preceding claims than a third generation oral contraceptive, said compound or composition not being a second generation oral contraceptive.

27. A sex steroid compound or composition according to claim 26 characterised by a lower increase in APR level or equivalent increase in APR level as determined in a manner according to any of the claims 1-26 than a second generation oral contraceptive.

28. A sex steroid compound or composition according to claim 26 or 27 characterised by no increase in APR level as determined in a manner according to any of the claims 1-26.

29. A sex steroid compound or composition according to any of claims 26-28 in a form suitable for oral application.

30. A sex steroid compound or composition according to any of claims 26-28 in a form other than for oral application, said compound or composition excluding Progestasert, LNG-20, Norplant, Depot-medroxyprogesterone acetate (Depo Provera) in the form of an injection, norethindrone enanthate in the form of an injection and oestragen in a transdermally applicable form.

31. A sex steroid formulation according to claim 30 in the form of an intrauterinary device.

32. A sex steroid formulation according to claim 30 in the form of an vaginally applicable form.

33. A sex steroid formulation according to claim 30 in the form of an transdermally applicable form, such as a patch, plaster or cream.

34. A sex steroid formulation according to claim 30 in the form of a subcutaneously applicable form.

35. A sex steroid formulation according to any of the claims 26-34 comprising an estrogenic variant that does not stimulate the acute phase reactants.

36. A sex steroid formulation according to any of the claims 26-35 comprising a progestogen variant or other compound that inhibits the acute phase activation of estrogen and/or inhibits the basic acute phase reaction and preferably at least to a degree sufficient to negate the acute phase reaction.
